(19) United States
(12) Patent Application Publication
(10) Pub. No.: US 2021/0300889 A1
(43) Pub. Date: Sep. 30, 2021

(54) METHOD FOR PREPARING ARTIFICIALLY SYNTHETIC (R,S)-NICOTINE SALT

(71) Applicant: FEELLIFE HEALTH INC., Shenzhen (CN)

(72) Inventors: Jian HUA, Shenzhen (CN); Quanfu LV, Shenzhen (CN)

(21) Appl. No.: 17/151,326

(22) Filed: Jan. 18, 2021

(30) Foreign Application Priority Data
Mar. 25, 2020 (KR) 10-2020-0036097

(51) Int. Cl. C07D 401/04

(52) U.S. Cl. CPC C07D 401/04

(57) ABSTRACT

The present invention discloses a method for preparing an artificially synthetic (R,S)-nicotine salt, comprising: S1, reacting 4-methylamino-1-(3-pyridine)-butanone hydrochloride and an alkaline substance at −5 to 5° C.; S2, concentrating a reactant obtained in step S1, and adding a first refining solvent for refining to obtain 1-methyl-2-(3-pyridine)-2-pyrrolidinol; S3, adding a reducing agent into 1-methyl-2-(3-pyridine)-2-pyrrolidinol, and reacting at 15 to 35° C.; and S4, concentrating a reactant obtained in step S3, adding a second refining solvent for refining, and then adding an acid for reaction, to obtain the artificially synthetic (R,S)-nicotine salt. The present invention innovatively proposes a two-step method to synthesize the (R,S)-nicotine salt, and the prepared (R,S)-nicotine salt does not contain any other harmful tobacco compounds. The method of the present invention is simple in process, high in product, and suitable for large-scale industrial production.

### CROSS-REFERENCE TO RELATED APPLICATIONS

The present application claims the benefit of and priority to Korean Patent Application No. 10-2020-0036097, filed on Mar. 25, 2020, which is incorporated herein by reference.

### FIELD OF THE INVENTION

The present invention relates to a method for preparing an artificially synthetic (R,S)-nicotine salt, in particular to a method for preparing a (R,S)-nicotine salt from 4-methylamino-1-(3-pyridine)-butanone hydrochloride through a two-step reaction.

### BACKGROUND OF THE INVENTION

The disclosure of the background is only for the purpose of increasing the understanding of the general background of the present invention and is not necessarily to be taken as an acknowledgement or in any way that this information constitutes a prior art that is already well known to a person skilled in the art.

Nicotine, also known as nicotiniod, is an alkaloid existing in Solanaceae plants, and is an important component of tobacco. Nicotine is a typical agonist of nicotinic acetylcholine receptors which have important regulatory effects on a central nervous system. Studies have shown that nicotine is expected to be a effective drug for treating Parkinson's disease, Alzheimer's disease, schizophrenia, epilepsy and depression. At present, the nicotine available in the market is the one mainly extracted from tobacco plants, which is affected by many factors such as raw materials, climates and cycles. Moreover, when nicotine is extracted from tobacco plants, more other impurities that are harmful to human body will also be extracted, such as anatabine, nicotyrine, cotinine, myosmine, nicotine-N-oxide, nornicotine and neonicotine, mentioned in the United States Pharmacopoeia, European Pharmacopoeia and British Pharmacopoeia. In addition, racemic nicotine (R,S-nicotine) and nicotine (S-nicotine) extracted from tobacco plants have essentially similar pharmacological activity. The racemic nicotine (R,S-nicotine) is slightly slower than nicotine (S-nicotine) extracted from tobacco plants only in acting time, but has much lower toxicity. (R,S)-nicotine and (R,S)-nicotine salts can only be obtained by synthesis, and the artificially synthetic (R,S)-nicotine and artificially synthetic (R,S)-nicotine salts prepared by the present invention do not contain any other harmful tobacco compounds.

The literature, Journal of Organic Chemistry, 1990, 55 (6), 1736-44, reported synthesis of racemic nicotine starting from pyrrolidine through a four-step reaction, as shown in reaction formula 1.

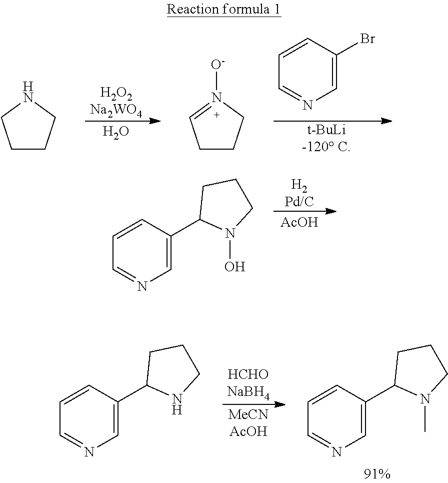

In the literature, the tert-butyl lithium and the low reaction temperature of −120° C. increase the difficulty of industrial production, and the yield of this method is low.

The literature, Journal of the Chemical Society, Perkin Transactions, 2002 (2), 143-154, reported a method for preparing racemic nicotine through a four-step reaction starting from nicotinic acid, as shown in reaction formula 2.

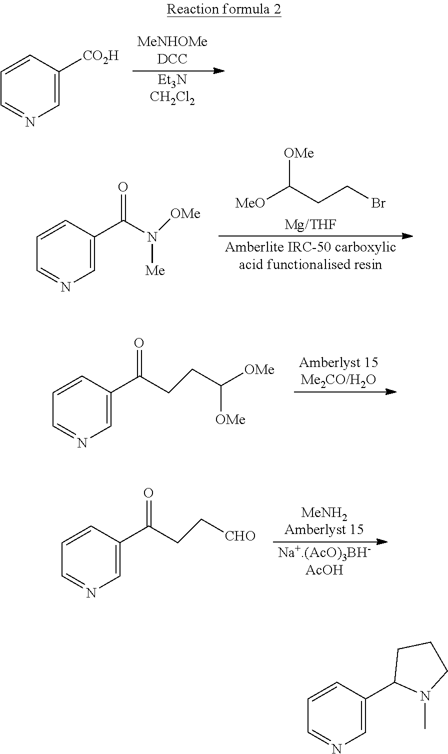

In this literature, the Grignard reagent used also limits the application of this method in industrialization.

Further, literature, Synlett, 2009 (15), 2497-2499, reported the preparation of racemic nicotine with 3-pyridinecarboxaldehyde as a starting material, as shown in reaction formula 3.

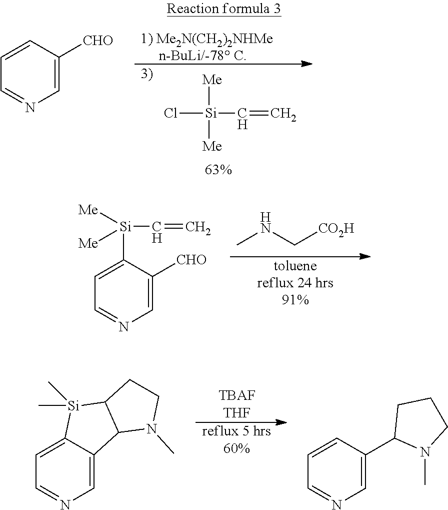

Similar to the above literatures, duce to the low reaction temperature of −78° C., this method still cannot fundamentally overcome the problem that racemic nicotine is difficultly industrially produced.

Further, the literature, Journal of Heterocyclic Chemistry, 2009, 46 (6), 1252-1258, reported a method for preparing racemic nicotine, as shown in reaction formula 4.

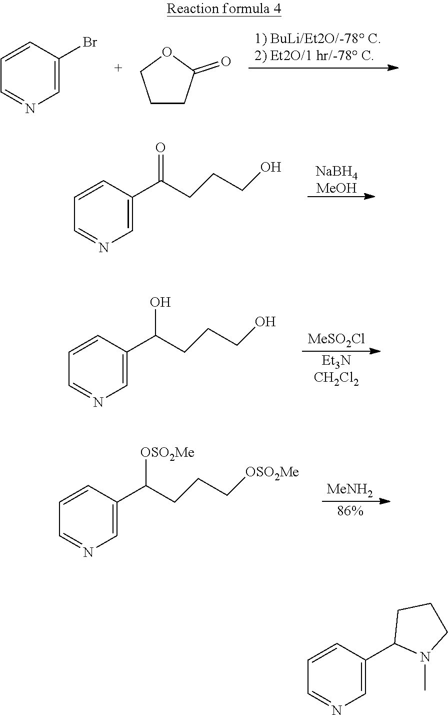

In this literature, metal exchange of 3-bromopyridine is performed by using butyllithium at low temperature, which also has the disadvantage that large-scale production cannot be achieved.

In conclusion, the existing methods for preparing racemic nicotine not only use expensive reagents, but also often react at low-temperature, have the disadvantages of many steps, long reaction cycles and increased costs, and therefore are difficultly industrially produced.

### SUMMARY OF THE INVENTION

The present invention aims to at least solve one of the technical problems existing in the prior art. To this end, the present invention innovatively proposes a method for preparing an artificially synthetic (R,S)-nicotine salt. The obtained artificially synthetic (R,S)-nicotine salt does not contain any other harmful tobacco compounds. This method has the advantages of simple process, low cost, high purity, simple operation, green and environmental protection, which is suitable for large-scale industrial production.

In the first aspect, the present invention provides a method for preparing an artificially synthetic (R, S)-nicotine salt, comprising the following steps:

S1, reacting 4-methylamino-1-(3-pyridine)-butanone hydrochloride with an alkaline substance at -5 to 5° C.;

S2, concentrating a reactant obtained in step S1, adding a first refining solvent for refining to obtain 1-methyl-2-(3-pyridine)-2-pyrrolidinol;

S3, adding a reducing agent into 1-methyl-2-(3-pyridine)-2-pyrrolidinol, and reacting at 15 to 35° C.; and

S4, concentrating a reactant obtained in step S3, adding a second refining solvent for refining, and then adding an acid for reaction, to obtain the artificially synthetic (R,S)-nicotine salt.

The method for preparing the artificially synthetic (R,S)-nicotine salt according to the embodiment of the present invention at least has the following beneficial effects:

In the method for preparing the artificially synthetic (R,S)-nicotine salt provide in the embodiment of the present invention, 4-methylamino-1-(3-pyridine)-butanone is free from 4-methylamino-1-(3-pyridine)-butanone hydrochloride under an alkaline condition, and then 4-methylamino-1-(3-pyridine)-butanone is subjected to condensation reaction by a nitrogen atom attacking a carbon atom on carbonyl under the alkaline condition to generate an intermediate 1-methyl -2-(3-pyridine)-2-pyrrolidinol. The principle of reaction is shown as follows:

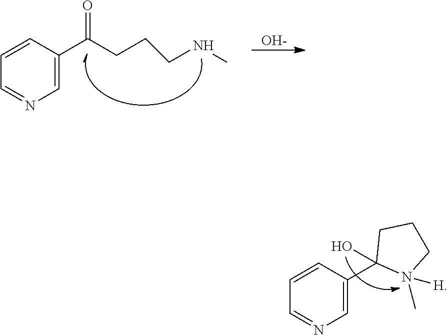

Then, the intermediate undergoes a reduction reaction with a reducing agent. The reason and purpose for adding the reducing agent are as follows: the intermediate 1-methyl-2-(3-pyridine)-2-pyrrolidinol is dehydrated under the alkaline condition to form a double bond and generate 1-methyl-2-(3-pyridine)-2-pyrrolidinene, and the added reducing agent can reduce the double bond in 1-methyl-2-(3-pyridine)-2-pyrrolidinene, so as to generate 1-methyl-2-(3-pyridine)-2-pyrrolidine, namely obtain (R,S)-nicotine. The principle of reaction is shown as follows:

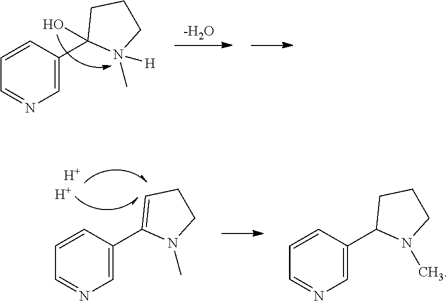

The generated (R,S)-nicotine reacts with acid to form a (R,S)-nicotine salt. The preparation method provided by the embodiment of the present invention does not require the addition of Grignard reagents such as tert-butyllithium and is mild in reaction condition, avoids the problems that the conventional addition of Grignard reagents requires harsh reaction conditions. At the same time, this method is simple in process, low in cost, simple in operation, green and environmentally friendly, and suitable for large-scale industrial production, and has more application values in industrial production.

In the embodiments of the present invention, refining means extraction or steam distillation. The method for preparing the artificially synthetic (R, S)-nicotine salt according to some embodiments of the present invention, comprises the following steps:

S1, adding 4-methylamino-1-(3-pyridine)-butanone hydrochloride, a solvent and an alkaline substance with a concentration of 0.1 molar concentration ˜10 molar concentration into a reaction vessel, and reacting at a low temperature of −5 to 5° C.;

S2, concentrating a reactant obtained in the above reaction, refining a concentrate with the first refining solvent to obtain 1-methyl-2-(3-pyridine)-2-pyrrolidinol;

S3, adding 1-methyl-2-(3-pyridine)-2-pyrrolidinol and a solvent into a reaction vessel, and adding an appropriate amount of a reducing agent for reaction at a temperature of 15 to 35° C.; and

S4, concentrating a reactant obtained in step S3, and refining a concentrate with a second refining solvent to obtain (R,S)-nicotine, and then reacting (R,S)-nicotine with a suitable acid, and refining a reaction product with a solvent to obtain the synthetic (R, S)-nicotine salt.

In the embodiments of the present invention, the molar concentration means mol/L. According to some embodiments of the present invention, the solvents used in step S1 and step S3 are independently selected from one or a mixture of any several of water, propylene glycol, methanol, ethanol, glycerol, propanol, isopropanol, tert-butanol and ethylene glycol.

According to some embodiments of the present invention, in step S4, in the steps of “refining a concentrate with a second refining solvent” and “refining a reaction product with a solvent”, the solvents used in refining are independently selected from one or a mixture of any several of water, methanol, ethanol, diethyl ether, petroleum ether, ethyl acetate, n-hexane and tetrahydrofuran.

According to some embodiments of the present invention, in step S1, the pH of the reaction system is adjusted to 7.5 to 10.0 by adding the alkaline substance. In the embodiment of the present invention, the pH of the reaction system is adjusted to 7.5 to 10.0 by using alkaline substance. Experiments show that when the pH is 7.5 to 10.0, the conversion rate of the product is high and the byproducts are few.

According to some embodiments of the present invention, in step S1, the alkaline substance is any one of sodium hydroxide, potassium hydroxide, sodium carbonate, ammonia, triethylamine, potassium carbonate, sodium bicarbonate, potassium bicarbonate and triphenylphosphorus.

According to some embodiments of the present invention, in step S3, the reducing agent is any one of hydrogen, stannous chloride, sodium borohydride, potassium borohydride and lithium aluminium tetrahydride.

According to some embodiments of the present invention, in step S2, the first refining solvent is one or a mixture of any several of water, petroleum ether, methanol, ethanol, diethyl ether, isopropanol and ethyl acetate.

According to some embodiments of the present invention, in step S4, the acid is an organic acid or an inorganic acid.

According to some embodiments of the present invention, the organic acid is any one of formic acid, acetic acid, propionic acid, butyric acid, valeric acid, caproic acid, caprylic acid, capric acid, citric acid, phenylacetic acid, benzoic acid, pyruvic acid, lactic acid, tartaric acid, salicylic acid, sorbic acid or malic acid; the inorganic acid is any one of hydrochloric acid, sulfuric acid, phosphoric acid or oxalic acid.

### BRIEF DESCRIPTION OF THE DRAWINGS

The accompanying drawings formed a part of the present application are provided to further understand the present application, the illustrative embodiments of the present application and descriptions thereof are used to explain the present application and are not intended to inappropriately limit thereto.

FIG. 1 is a mass spectrum of (R,S)-nicotine synthesized according to an embodiment of the present invention.

FIG. 2 is a chiral analysis high performance liquid chromatography (HPLC) chromatogram of (R,S)-nicotine synthesized according to an embodiment of the present invention.

### DETAILED DESCRIPTION OF THE EMBODIMENTS

In the following, the concept of the present invention and the technical effects obtained will be clearly and completely described with reference to the embodiments of the present application to fully understand the purpose, features and effects of the present invention. Obviously, the described embodiments are only a part of the embodiments of the present invention, rather than all the embodiments. Based on the embodiments of the present invention, other embodiments obtained by a person skilled in the art without creative efforts all belong to the protection scope of the present invention.

The solvents or reagents used in the following embodiments are all produced by Sinopharm Chemical Reagent Co., Ltd.; the optical rotation is measured by a WZZ-2A model polarimeter; the melting point is measured by an MP70 model melting point apparatus; the ultraviolet ray is measured by a UV2550 model ultraviolet spectrophotometer; the proton nuclear magnetic resonance spectroscopy is measured on a Varian Mercury 500 instrument; the mass spectrum is measured with an API3000 model mass spectrometer. All spectra are consistent with the inferred structure, and characteristic peaks are expressed by conventional abbreviations: S, singlet; D, doublet; T, triplet; Q, quartet; M, multiplet.

The 4-methylamino-1-(3-pyridine)-butanone hydrochloride used in the following embodiments refers to 4-methylamino-1-(3-pyridine)-butanone dihydrochloride.

### Embodiment 1

This embodiment provides an artificially synthetic (R, S)-nicotine salt, which was prepared by the following steps:

S1, dissolving 4-methylamino-1-(3-pyridine)-butanone hydrochloride (13.8 g, 0.055 mol) into 160 mL of water, adjusting the pH of the reaction system with 5 mol/L potassium hydroxide or sodium hydroxide at −5° C. to be slightly alkaline(pH 8), and reacting for 5 hours under stirring;

S2, concentrating a reactant obtained in step S1, and refining a concentrate with a mixture of water and methanol to obtain an intermediate 1-methyl-2-(3-pyridine)-2-pyrrolidinol;

S3, dissolving the intermediate with 180 mL of a mixture of water and ethanol, adding sodium borohydride (3.8 g) at −5° C., and heating to 15° C., and reacting for 2 hours under stirring;

S4, extracting the above reaction product with ethyl acetate, drying, and concentrating to obtain a light yellow oily crude product, adding 110 mL of water for direct distillation, extracting the water with 270 mL of n-hexane or tetrahydrofuran, drying, and concentrating to obtain 7.9 g of oily product. The chirality was measured with high performance liquid chromatography, and ratios of chiral R configuration and S configuration in the measured product were almost equal. The optical rotation of the product was measured by the polarimeter to be zero, indicating that the product was (R,S)-nicotine with a yield of 72%. The purity of (R, S)-nicotine in the oily product was determined to be 99.2% by HPLC. Citric acid was added into the oily product and stirred evenly to obtain the (R,S)-nicotine salt.

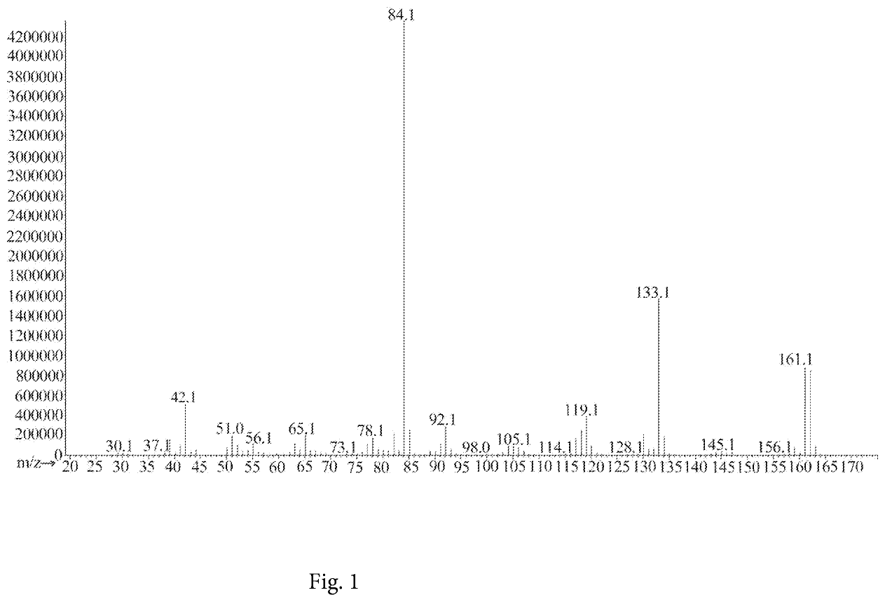

The mass spectrum of the (R, S)-nicotine synthesized in this embodiment was shown in FIG. 1. The artificially synthetic (R,S)-nicotine and (R,S)-nicotine salt prepared in the embodiment of the present invention have high purity and do not contain any other harmful tobacco compounds. the present invention is simple to operate, high in purity, mild in reaction conditions, green and environmentally friendly, few in steps, and suitable for large-scale industrial production.

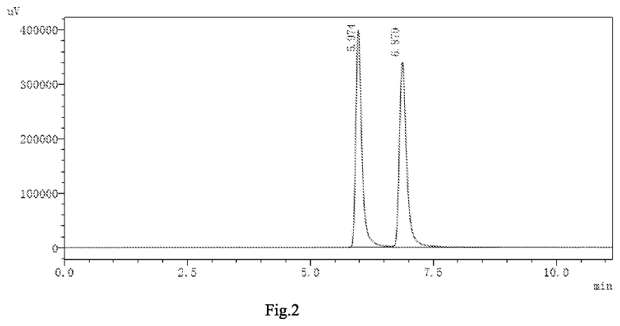

The chiral chromatogram of (R, S)-nicotine synthesized in this embodiment was shown in FIG. 2, and the results were shown in Table 1. The ratios of chiral R configuration and S configuration in the artificially synthetic (R, S)-nicotine prepared in the embodiment of the present invention were almost equal, which was consistent with the result of the optical rotation of the product being measured by the polarimeter to be zero.

TABLE 1
Detector A Ch2 254 nm

| S# | Retention Time | Height | Area % | Resolution | Tailing Factor |
|---|---|---|---|---|---|
| 1 | 5.974 | 400000 | 49.643 | — | 1.518 |
| 2 | 6.870 | 339524 | 50.357 | 3.683 | 1.480 |

### Embodiment 2

This embodiment provides an artificially synthetic (R,S)-nicotine salt, which was prepared by the following steps:

S1, dissolving 4-methylamino-1-(3-pyridine)-butanone hydrochloride (25.1 g, 0.1 mol) into 150 mL of water and methanol, adjusting the pH to 8.5 with sodium carbonate or potassium carbonate at 0° C., and reacting for 3 hours under stirring;

S2, concentrating a reactant obtained in step S1, and refining a concentrate with a mixture of water and methanol to obtain an intermediate 1-methyl-2-(3-pyridine)-2-pyrrolidinol;

S3, dissolving the intermediate with 160 mL of methanol, adding lithium aluminium tetrahydride (3.7 g, 0.1 mol) at 0° C., heating to 25° C., and reacting for 2 hours under stirring;

S4, extracting the above reaction product with methanol, drying, and concentrating to obtain a light yellow oily crude product, adding 210 mL of water for direct distillation, extracting the water with 290 mL of petroleum ether or diethyl ether, drying, and concentrating to obtain 12.5 g of oily product. The optical rotation was measured to be zero, indicating that the product was (R,S)-nicotine. The purity of (R,S)-nicotine was determined to be 99.5% by HPLC. Oxalic acid was added into the oily product and stirred evenly to obtain the (R,S)-nicotine salt.

### Embodiment 3

This embodiment provides an artificially synthetic (R, S)-nicotine salt, which was prepared by the following steps:

S1, dissolving 4-methylamino-1-(3-pyridine)-butanone hydrochloride (25.1 g, 0.1 mol) with 165 mL of water and ethanol, adjusting the pH to 7.5 with sodium bicarbonate or potassium bicarbonate at 5° C., and reacting for 2 hours under stirring;

S2, concentrating a reactant obtained in step S1, and refining a concentrate with a mixture of water and methanol to obtain an intermediate 1-methyl-2-(3-pyridine)-2-pyrrolidinol;

S3, dissolving the intermediate with 180 mL of propanol or isopropanol, adding potassium borohydride (5.4 g, 0.1 mol) at 5° C., heating to 25° C., and reacting for 2 hours under stirring;

S4, adding 200 mL of distilled water for steam distillation, extracting the distilled water phase with 350 mL of ethyl acetate, and concentrating the ethyl acetate to dryness to obtain 12.0 g of light yellow oil. The optical rotation was measured to be zero, indicating that the product was (R,S)-nicotine. The purity of (R,S)-nicotine was determined to be 99.5% by HPLC. Acetic acid was added into the oily product and stirred evenly to obtain the (R,S)-nicotine salt.

### Embodiment 4

This embodiment provides an artificially synthetic (R,S)-nicotine salt, which was prepared by the following steps: S1, dissolving 4-methylamino-1-(3-pyridine)-butanone hydrochloride (25.1 g, 0.1 mol) into 160 mL of water and propanol or glycerol, adjusting the pH to 8.5 with ammonia at 0° C., and t reacting for 3 hours under stirring;

S2, concentrating a reactant obtained in step S1, and refining a concentrate with a mixture of water and ethyl acetate to obtain an intermediate 1-methyl-2-(3-pyridine)-2-pyrrolidinol;

S3, dissolving the intermediate with 170 mL of a mixture of water and ethanol, adding stannous chloride (18.9 g, 0.1 mol) at 0° C., heating to 35° C., and reacting for 2 hours under stirring;

S4, extracting the above reaction product with petroleum ether, drying, and concentrating to obtain a light yellow oily crude product, adding 210 mL of water for direct distillation, extracting the water with 200 mL of ethyl acetate, drying, and concentrating to obtain 12.5 g of oily product. The optical rotation was measured to be zero, indicating that the product was (R,S)-nicotine. The purity of (R, S)-nicotine was determined to be 99.5% by HPLC. Malic acid was added into the oily product and stirred evenly to obtain the (R,S)-nicotine salt.

### Embodiment 5

This embodiment provides an artificially synthetic (R,S)-nicotine salt, which was prepared by the following steps:

S1, dissolving 4-methylamino-1-(3-pyridine)-butanone hydrochloride (25.1 g, 0.1 mol) with 160 mL of water and isopropanol, adjusting the pH to 7.5 with triethylamine at 5° C., and reacting for 2 hours under stirring;

S2, concentrating a reactant obtained in step S1, and refining a concentrate with an isopropanol mixture to obtain an intermediate 1-methyl-2-(3-pyridine)-2-pyrrolidinol;

S3, dissolving the intermediate with 175 mL of ethylene glycol, adding stannous chloride (18.9 g, 0.1 mol) at 5° C., heating to 25° C., and reacting for 2 hours under stirring;

S4, adding 200 mL of distilled water for steam distillation, extracting the distilled water phase with 350 mL of ethanol or methanol, and concentrating the ethanol or methanol to dryness to obtain 12.0 g of light yellow oil. The optical rotation was measured to be zero, indicating that the product was (R, S)-nicotine. The purity of (R, S)-nicotine was determined to be 99.5% by HPLC. Lactic acid was added into the oily product and stirred evenly to obtain the (R, S)-nicotine salt.

### Embodiment 6

This embodiment provides an artificially synthetic (R,S)-nicotine salt, which was prepared by the following steps: S1, dissolving 4-methylamino-1-(3-pyridine)-butanone hydrochloride (25.1 g, 0.1 mol) into 165 mL of water and tert-butanol, adjusting the pH to 8.5 with triphenylphosphorus at 0° C., and reacting for 3 hours under stirring;

S2, concentrating a reactant obtained in step S1, and refining a concentrate with a mixture of water and diethyl ether or petroleum ether to obtain an intermediate 1-methyl-2-(3-pyridine)-2-pyrrolidinol;

S3, dissolving the intermediate with 180 mL of tert-butanol, adding potassium borohydride (5.4 g, 0.1 mol) at 0° C., heating to 25° C., and reacting for 2 hours under stirring;

S4, extracting the above reaction product with n-hexane, drying, and concentrating to obtain a light yellow oily crude product, adding 210 mL of water for direct distillation, extracting the water with 300 mL of methanol, drying, and concentrating to obtain 12.5 g of oily product. The optical rotation was measured to be zero, indicating that the product was (R,S)-nicotine. The purity of (R,S)-nicotine was determined to be 99.5% by HPLC. Valeric acid was added into the oily product and stirred evenly to obtain the (R, S)-nicotine salt.

### Embodiment 7: Synthesis of (R,S)-Nicotine Salt

S1, dissolving 4-methylamino-1-(3-pyridine)-butanone hydrochloride (25.1 g, 0.1 mol) into 155 mL of methanol, adjusting the pH to 8.5 with sodium carbonate or potassium carbonate at 0° C., and reacting for 3 hours under stirring;

S2, concentrating a reactant obtained in step S1, and refining a concentrate with a mixture of water and methanol to obtain an intermediate 1-methyl-2-(3-pyridine)-2-pyrrolidinol;

S3, dissolving the intermediate with 165 mL of methanol, adding lithium aluminium tetrahydride (3.7 g, 0.1 mol) at 0° C., heating to 25° C., and reacting for 2 hours under stirring;

S4, extracting the above reaction product with methanol, drying and concentrating to obtain a light yellow oily crude product, adding 215 mL of water for direct distillation, extracting the water with 300 mL of diethyl ether, drying and concentrating to obtain 12.5 g of oily product. The optical rotation was measured to be zero, indicating that the product was (R,S)-nicotine. The purity of (R,S)-nicotine was determined to be 99.8% by HPLC. Tartaric acid was added into the oily product and stirred evenly to obtain the (R,S)-nicotine salt.

### Embodiment 8: Synthesis of (R,S)-Nicotine Salt

S1, dissolving 4-methylamino-1-(3-pyridine)-butanone hydrochloride (25.1 g, 0.1 mol) with 160 mL of isopropanol, adjusting the pH to 7.5 with triethylamine at 5° C., and reacting for 2 hours under stirring;

S2, concentrating a reactant obtained instep S1, and refining a concentrate with an isopropanol mixture to obtain an intermediate 1-methyl-2-(3-pyridine)-2-pyrrolidinol;

S3, dissolving the intermediate with 175 mL of ethylene glycol, adding stannous chloride (18.9 g, 0.1 mol) at 5° C., heating to 25° C., and reacting for 2 hours under stirring;

S4, adding 200 mL of distilled water for steam distillation, extracting the distilled water phase with 350 mL of ethanol or methanol, and concentrating the ethanol or methanol to dryness to obtain 12.0 g of light yellow oily product. The optical rotation was measured to be zero, indicating that the product was (R,S)-nicotine. The purity of (R,S)-nicotine was determined to be 99.8% by HPLC. Phosphoric acid was added into the oily product and stirred evenly to obtain the (R,S)-nicotine salt.

### Embodiment 9: Synthesis of (R,S)-Nicotine Salt

S1, dissolving 4-methylamino-1-(3-pyridine)-butanone hydrochloride (25.1 g, 0.1 mol) with 160 mL of water and propanol or glycerol, adjusting the pH to 8.5 with ammonia at 0° C., and reacting for 3 hours under stirring;

S2, concentrating a reactant obtained in step S1, and the refining a concentrate with a mixture of water and ethyl acetate to obtain an intermediate 1-methyl-2-(3-pyridine)-2-pyrrolidinol;

S3, dissolving the intermediate with 170 mL of a mixture of water and ethanol, adding sodium borohydride (3.8 g, 0.1 mol) at 0° C., heating to 35° C., and reacting for 2 hours under stirring;

S4, extracting the above reaction product with petroleum ether, drying and concentrating to obtain a light yellow oily crude product, adding 210 mL of water for direct distillation, extracting the water with 200 mL of ethyl acetate, drying and concentrating to obtain 12.5 g of oily product. The optical rotation was measured to be zero, indicating that the product was (R, S)-nicotine.

The purity of (R,S)-nicotine was determined to be 99.8% by HPLC. Lactic acid was added into the oily product and stirred evenly to obtain the (R,S)-nicotine salt.

### Embodiment10: Synthesis of (R,S)-Nicotine Salt

S1, dissolving 4-methylamino-1-(3-pyridine)-butanone hydrochloride (25.1 g, 0.1 mol) with 155 mL of methanol, adjusting the pH to 8.5 with sodium carbonate or potassium carbonate at 0° C., and reacting for 3 hours under stirring;

S2, concentrating a reactant obtained in step S1, and refining a concentrate with a mixture of water and methanol to obtain an intermediate 1-methyl-2-(3-pyridine)-2-pyrrolidinol;

S3, dissolving the intermediate with 165 mL of methanol, adding lithium aluminium tetrahydride (3.7 g, 0.1 mol) at 0° C., heating to 25° C., and reacting for 2 hours under stirring;

S4, extracting the above reaction product with methanol, drying and concentrating to obtain a light yellow oily crude product, adding 215 mL of water for direct distillation, extracting the water with 300 mL of diethyl ether, drying, and concentrating to obtain 12.5 g of oily product. The optical rotation was measured to be zero, indicating that the product was (R,S)-nicotine. The purity of (R, S)-nicotine was determined to be 99.8% by HPLC. 6.25 g of tartaric acid was added into the oily product, stirred evenly, then refined with a mixture of diethyl ether and ethanol to obtain 11.8 g of (R,S)-nicotine salt.

### Embodiment 11: Synthesis of (R, S)-Nicotine Salt

S1, dissolving 4-methylamino-1-(3-pyridine)-butanone hydrochloride (25.1 g, 0.1 mol) with 160 mL of isopropanol, adjusting the pH to 7.5 with triethylamine at 5° C., and reacting for 2 hours under stirring;

S2, concentrating a reactant obtained in step S1, and refining a concentrate with an isopropanol mixture to obtain an intermediate 1-methyl-2-(3-pyridine)-2-pyrrolidinol;

S3, dissolving the intermediate with 175 mL of ethylene glycol, adding stannous chloride (18.9 g, 0.1 mol) at 5° C., heating to 25° C., and reacting for 2 hours under stirring;

S4, adding 200 mL of distilled water for steam distillation, extracting the distilled water phase with 350 mL of ethanol or methanol, and concentrating the ethanol or methanol to dryness to obtain 12.0 g of light yellow oily product. The optical rotation was measured to be zero, indicating that the product was (R,S)-nicotine. The purity of (R,S)-nicotine was determined to be 99.8% by HPLC. 5.0 g of phosphoric acid was added into the oily product, stirred evenly, then refined with a mixture of n-hexane and water and ethanol to obtain 11.5 g of (R,S)-nicotine salt.

### Embodiment 12: Synthesis of (R, S)-Nicotine Salt

S1, dissolving 4-methylamino-1-(3-pyridine)-butanone hydrochloride (25.1 g, 0.1 mol) into 160 mL of water and propanol or glycerol, adjusting the pH to 8.5 with ammonia at 0° C., and reacting for 3 hours under stirring;

S2, concentrating a reactant obtained in step S1, and refining a concentrate with a mixture of water and ethyl acetate to obtain an intermediate 1-methyl-2-(3-pyridine)-2-pyrrolidinol;

S3, dissolving the intermediate with 170 mL of a mixture of water and ethanol, adding sodium borohydride (16.2 g, 0.1 mol) at 0° C., heating to 35° C., and reacting for 2 hours under stirring;

S4, extracting the above reaction product with petroleum ether, drying and concentrating to obtain a light yellow oily crude product, adding 210 mL of water for direct distillation, extracting the water with 200 mL of ethyl acetate, drying, and concentrating to obtain 12.5 g of oily product. The optical rotation was measured to be zero, indicating that the product was (R, S)-nicotine. The purity of (R,S)-nicotine was determined to be 99.8% by HPLC. 12.5 g of phenylacetic acid was added into the oily product, stirred evenly, then refined with a mixture of ethanol and isopropanol to obtain 12.1 g of (R,S)-nicotine salt.

What is claimed is:

1. A method for preparing an artificially synthetic (R,S)-nicotine salt, comprising the following steps:
S1, reacting 4-methylamino-1-(3-pyridine)-butanone hydrochloride with an alkaline substance at −5 to 5° C.;
S2, concentrating a reactant obtained in step S1, adding a first refining solvent for refining to obtain 1-methyl-2-(3-pyridine)-2-pyrrolidinol;
S3, adding a reducing agent into 1-methyl-2-(3-pyridine)-2-pyrrolidinol, and reacting at 15 to 35° C.; and
S4, concentrating a reactant obtained in step S3, adding a second refining solvent for refining, and then adding an acid for reaction, to obtain the (R,S)-nicotine salt.

2. The method for preparing an artificially synthetic (R, S)-nicotine salt according to claim 1, comprising the following steps:
S1, adding 4-methylamino-1-(3-pyridine)-butanone hydrochloride, a solvent and an alkaline substance with a concentration of 0.1 molar concentration ˜10 molar concentration into a reaction vessel, and reacting at a low temperature of −5 to 5° C.;
S2, concentrating a reactant obtained in the above reaction, refining a concentrate with the first refining solvent to obtain 1-methyl-2-(3-pyridine)-2-pyrrolidinol;
S3, adding 1-methyl-2-(3-pyridine)-2-pyrrolidinol and a solvent into a reaction vessel, and then adding an appropriate amount of a reducing agent for reaction at a temperature of 15-35° C.; and
S4, concentrating a reactant obtained in step S3, and refining a concentrate with a second refining solvent to obtain (R,S)-nicotine, and then reacting (R,S)-nicotine with a suitable acid, and refining a reaction product with a solvent to obtain the synthetic (R,S)-nicotine salt.

3. The method for preparing an artificially synthetic (R,S)-nicotine salt according to claim 2, wherein the solvents used in step S1 and step S3 are independently selected from one or a mixture of any several of water, propylene glycol, methanol, ethanol, glycerol, propanol, isopropanol, tert-butanol and ethylene glycol.

4. The method for preparing an artificially synthetic (R,S)-nicotine salt according to claim 2, wherein in step S4, in the step of refining a concentrate with a second refining solvent and refining a reaction product with a suitable solvent, the solvents used in refining are independently selected from one or a mixture of any several of water, methanol, ethanol, diethyl ether, petroleum ether, ethyl acetate, n-hexane and tetrahydrofuran.

5. The method for preparing an artificially synthetic (R,S)-nicotine salt according to claim 1, wherein in step S1, the pH of the reaction system is adjusted to 7.5 to 10.0 by adding the alkaline substance.

6. The method for preparing an artificially synthetic (R,S)-nicotine salt according to claim 1, wherein in step S1, the alkaline substance is any one of sodium hydroxide, potassium hydroxide, sodium carbonate, ammonia, triethylamine, potassium carbonate, sodium bicarbonate, potassium bicarbonate and triphenylphosphorus.

7. The method for preparing an artificially synthetic (R,S)-nicotine salt according to claim 1, wherein in step S3, the reducing agent is any one of hydrogen, stannous chloride, sodium borohydride, potassium borohydride and lithium aluminium tetrahydride.

8. The method for preparing an artificially synthetic (R,S)-nicotine salt according to claim 1, wherein in step S2, the first refining solvent is one or a mixture of any several of water, petroleum ether, methanol, ethanol, diethyl ether, isopropanol and ethyl acetate.

9. The method for preparing an artificially synthetic (R,S)-nicotine salt according to claim 1, wherein in step S4, the acid is an organic acid or an inorganic acid.

10. The method for preparing an artificially synthetic (R,S)-nicotine salt according to claim 9, wherein the organic acid is any one of formic acid, acetic acid, propionic acid, butyric acid, valeric acid, caproic acid, caprylic acid, capric acid, citric acid, phenylacetic acid, benzoic acid, pyruvic acid, lactic acid, tartaric acid, salicylic acid, sorbic acid or malic acid; the inorganic acid is any one of hydrochloric acid, sulfuric acid, phosphoric acid or oxalic acid.
